(19) United States
(12) Patent Application Publication
(10) Pub. No.: US 2026/0273216 A1
(43) Pub. Date: Sep. 17, 2026

(54) A CONDUIT FOR A POSITIONING AND STABILISING STRUCTURE AND CONNECTOR FOR SAME

(71) Applicants: ResMed Pty Ltd, Bella Vista, New South Wales (AU); ResMed Asia Pte. Ltd., Singapore (SG)

(72) Inventors: Skye Kimberley SHARMA, Sydney (AU); Maximilian Aji WIJOYOSENO, Singapore (SG); Luke Emmanuel KLINKENBERG, Sydney (AU)

(21) Appl. No.: 19/469,309

(22) PCT Filed: Mar. 28, 2024
(86) PCT No.: PCT/AU2024/050286

§ 371 Date: Sep. 25, 2025

(30) Foreign Application Priority Data
Mar. 31, 2023 (AU) 2023900910
Mar. 27, 2024 (AU) 2024900836

(51) Int. Cl. A61M 16/06

(52) U.S. Cl. CPC A61M 16/0666; A61M 16/0683

(57) ABSTRACT

A patient interface including a positioning and stabilising structure with gas delivery tubes providing for flow of pressurised air from a connection port to a seal-forming structure around the patient's airways. The gas delivery tubes are covered by a textile material which is secured to the ends of same using parts of conduit connection structures that are also used to fluidly connect the tubes to the seal-forming structure and/or connection port.

### 1 CROSS-REFERENCE TO RELATED APPLICATIONS

This application claims priority to Australian Patent Application No. 2023910, filed 31 Mar. 2023 and Australian Patent Application No. 2024900836, filed 27 Mar. 2024, each of which is hereby incorporated by reference herein in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH2O relative to ambient pressure. For other forms of therapy, such as the delivery of oxygen, the patient interface may not include a seal sufficient to facilitate delivery to the airways of a supply of gas at a positive pressure of about 10 cmH2O. For flow therapies such as nasal HFT, the patient interface is configured to insufflate the nares but specifically to avoid a complete seal. One example of such a patient interface is a nasal cannula.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

To encourage patient compliance, the positioning and stabilising structure should be comfortable, easy to adjust and easy and intuitive to disconnect and/or doff. Areas which touch the patient's skin should not be hard or likely to scratch or otherwise irritate the skin.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

2.2.3.1.4 Pressurised Air Conduit used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

To encourage patient compliance, the headgear tubing may be comprised of one or more tubes of silicone or similar material which is covered with a textile for comfort and minimising of skin irritation.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

Another aspect of one form of the present technology comprises a positioning and stabilising structure to provide a force to hold a seal-forming structure in a therapeutically effective position on a patient's head, the seal-forming structure constructed and arranged to form a seal with a region of the patient's face surrounding an entrance to the patient's airways for sealed delivery of a flow of air at a therapeutic pressure of at least 6 cmH₂O above ambient air pressure throughout the patient's respirator cycle in use, the positioning and stabilising structure comprising:
at least one gas delivery tube to receive the flow of air from a connection port on top of the patient's head and an inferior end configured to deliver the flow of air to the entrance of the patient's airways via the seal-forming structure, the at least one gas delivery tube being constructed and arranged to contact, in use, at least a region of the patient's head superior to an otobasion superior of the patient's head, the at least one gas delivery tube comprising:
an inner tube comprising a tube wall defining a hollow interior for a flow path for air to flow from the connection port to the seal-forming structure, and wherein the inner tube is configured with an inferior end and a superior end;
an outer textile sleeve having an inferior end and a superior end; and
a conduit connection structure provided to at least one of the inferior and superior ends of the inner tube, wherein the conduit connection structure is configured to connect the inner tube to one of the connection port or seal-forming structure, wherein the conduit connection structure is configured with a first surface that forms part of the flow path through the hollow interior of the inner tube, and wherein the conduit connection structure is configured with a second surface away from the flow path through the hollow interior of the inner tube and forming at least a part of an attachment region;
wherein the attachment region is configured to receive one of the inferior and superior ends of the outer textile sleeve.

Another aspect of one form of the present technology comprises a gas delivery tube for a positioning and stabilising structure, wherein the positioning and stabilising structure is configured with a connection port on top of a patient's head and a seal forming structure constructed and arranged to form a seal with a region of the patient's face surrounding an entrance to the patient's airways for sealed delivery of a flow of air at a therapeutic pressure of at least 6 cmH₂O above ambient air pressure throughout the patient's respirator cycle in use, wherein the gas delivery tube is configured to receive the flow of air from the connection port to the seal-forming structure, the gas delivery tube comprising:
an inner tube comprising a tube wall defining a hollow interior for a flow path for air to flow from the connection port to the seal-forming structure, and wherein the inner tube is configured with an inferior end and a superior end;
an outer textile sleeve having an inferior end and a superior end; and
a conduit connection structure provided to at least one of the inferior and superior ends of the inner tube, wherein the conduit connection structure is configured to connect the inner tube to one of the connection port or seal-forming structure, wherein the conduit connection structure is configured with a first surface that forms part of the flow path through the hollow interior of the inner tube, and wherein the conduit connection structure is configured with a second surface away from the flow path through the hollow interior of the inner tube and forming at least a part of an attachment region;
wherein the attachment region is configured to receive one of the inferior and superior ends of the outer textile sleeve.

In examples, the conduit connection structure includes an engagement portion configured to engage with a complementary engagement portion provided to one of the connection port or seal-forming structure.

In examples, the conduit connection structure includes a first collar provided to one of the inferior end and superior end of the inner tube.

In examples, the first collar includes an inner side having first contact surface and a second contact surface, and an outer side.

In examples, the first contact surface of the first collar is substantially perpendicular to the second contact surface of the first collar.

In examples, the first contact surface contacts a mouth of the inner tube and the second contact surface contacts an interior surface of the hollow interior of the inner tube.

In examples, the attachment region is a portion of the second contact surface.

In examples, the attachment region is less than the length of the second contact surface.

In examples, the end of the second contact surface includes a ridge or protrusion.

In examples, the interior surface of the hollow interior of the inner tube includes a ridge or protrusion to abut that of the end of the second contact surface.

In examples, the inferior end of the textile sleeve is adhesively secured or otherwise bonded to the attachment region.

In examples, the inferior end of the textile sleeve is secured or otherwise bonded to the attachment region by overmoulding the first collar to one of the inferior end or superior end of the inner tube.

In examples, the inferior end of the textile sleeve is secured or otherwise bonded to the attachment region by overmoulding a second collar to one of the inferior end or superior end of the inner tube.

In examples, at least a portion of the outer side of the first collar is the first surface that forms at least part of the flow path through the hollow interior of the inner tube.

In examples, at least a portion of the first surface includes the engagement portion configured to engage with the complementary engagement portion provided to one of the connection port or seal-forming structure

In examples, the first collar includes an end cap.

In examples, the end cap is connected to the first collar on a surface opposing the first contact surface of the first collar.

In examples, the attachment region is the juncture between the end cap and the first collar.

In examples, the end cap is integrally formed with the first collar.

In examples, the juncture between the end cap and the first collar is a groove or recess.

In examples, the inferior end of the textile sleeve is adhesively secured or otherwise bonded to the attachment region.

In examples, the inferior end of the textile sleeve is secured or otherwise bonded to the attachment region by overmoulding the end cap to the first collar.

In examples, the conduit connection structure is provided to both the inferior end and superior end of the inner tube.

In examples, the conduit connection structure is comprised of one or more plastics material of polycarbonate (PCB), polypropylene (PPE), nylon or a blend thereof.

In examples, the inner tube is comprised of an elastomer.

In examples, the elastomer is comprised of silicone, thermoplastic elastomer (TPE), or thermoplastic polyurethane (TPU), or another elastomer material or a blend of same.

In examples, the outer textile sleeve is comprised of a thermofusible material.

In examples, the thermofusible material is a yarn.

In examples, one of the inferior end and superior end of the textile sleeve has been heat treated to form a lip.

In examples, the lip is received by the attachment region of the conduit connection structure.

In examples, both of the inferior end and superior end of the textile sleeve has been heat treated to form a lip.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

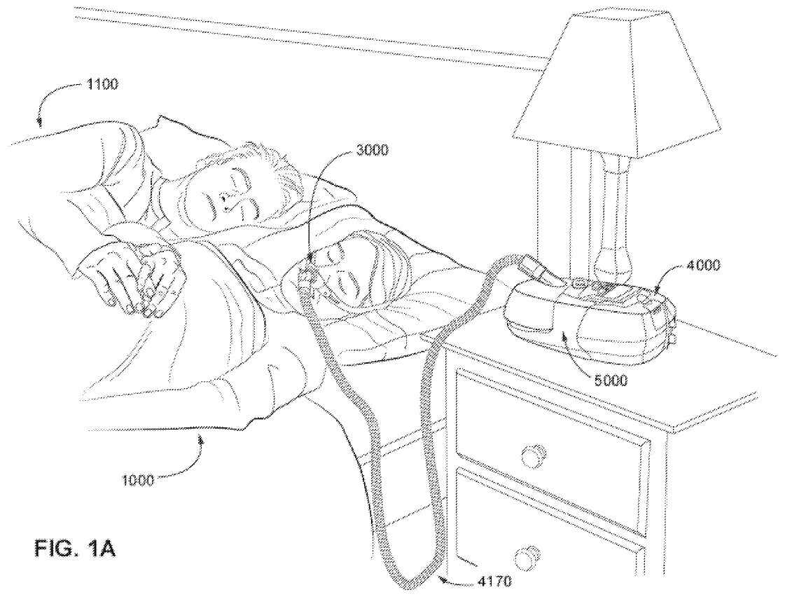

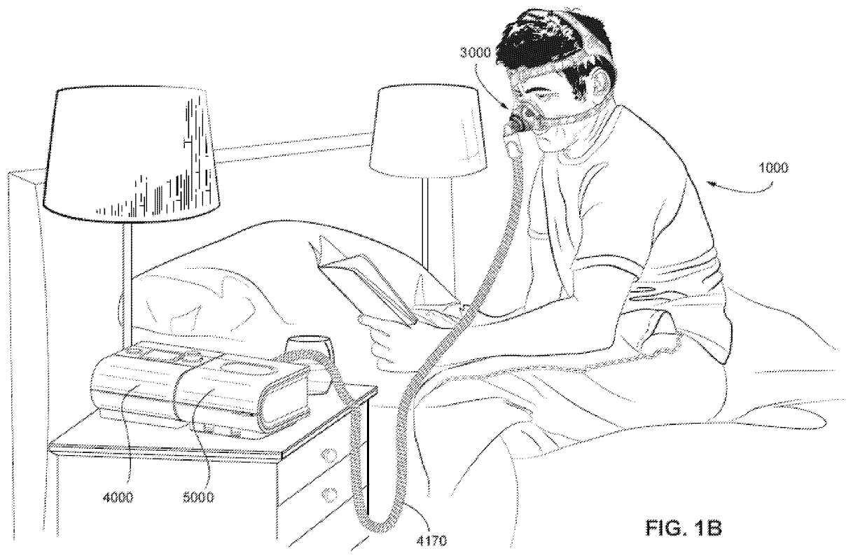

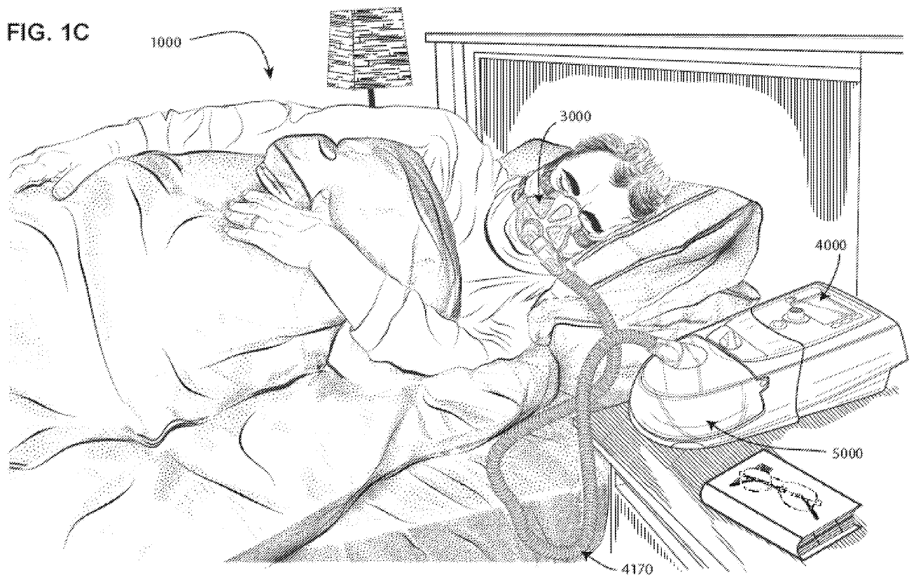

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

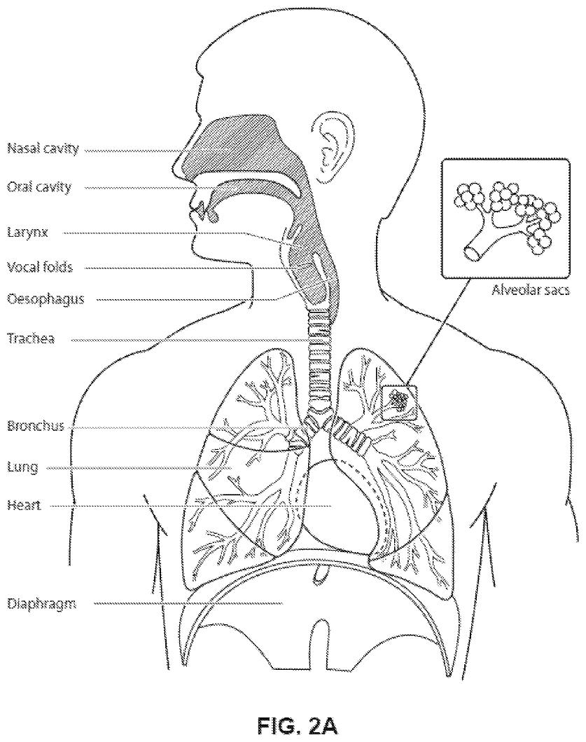

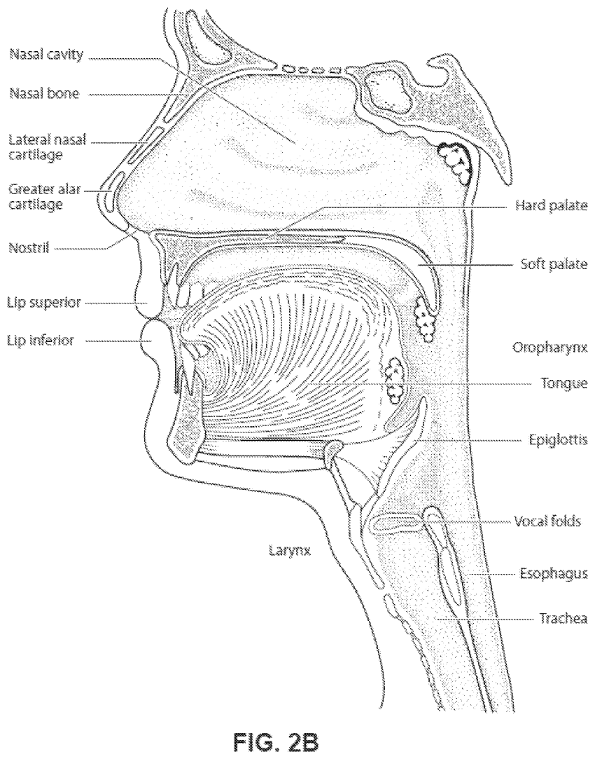

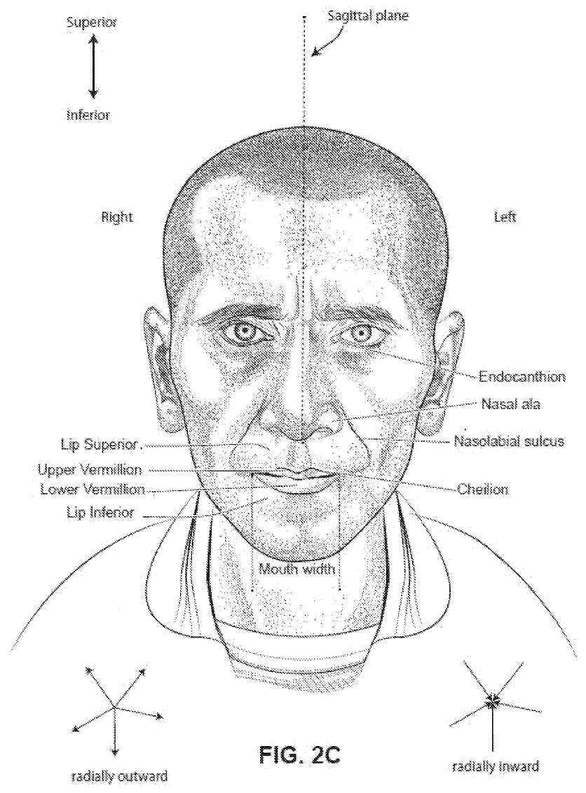

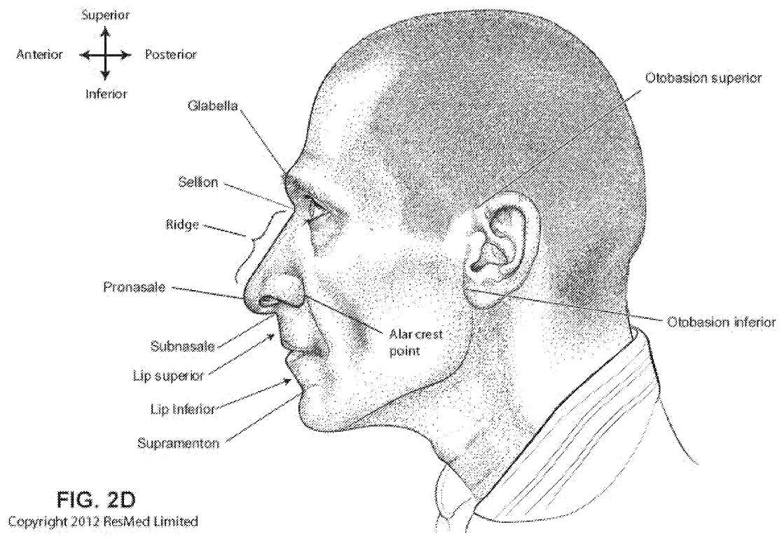

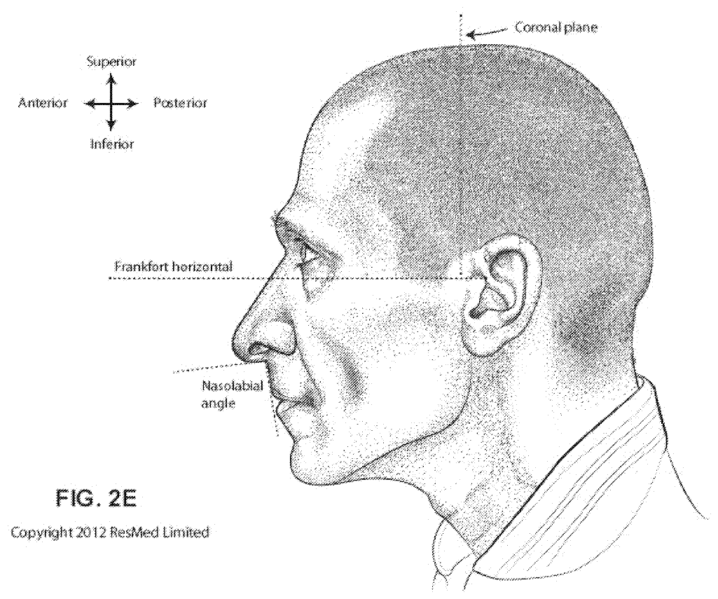

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

### 4.3 Patient Interface

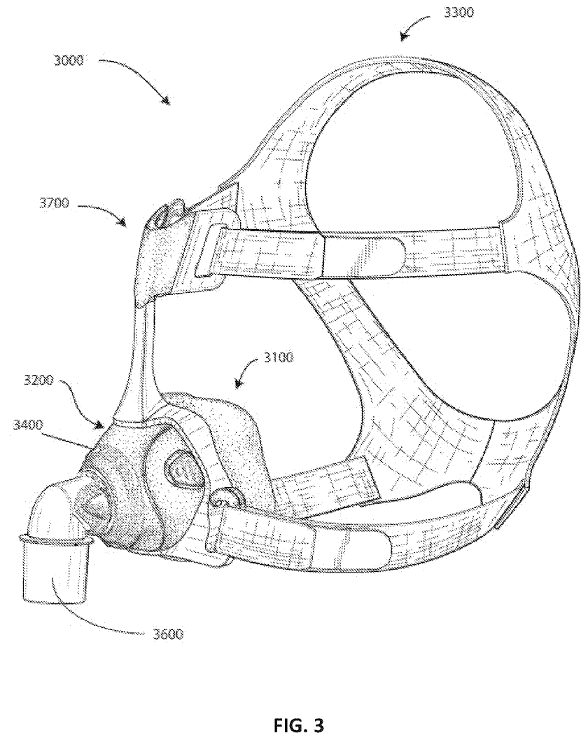

FIG. 3 shows a patient interface in the form of a nasal mask in accordance with one form of the present technology.

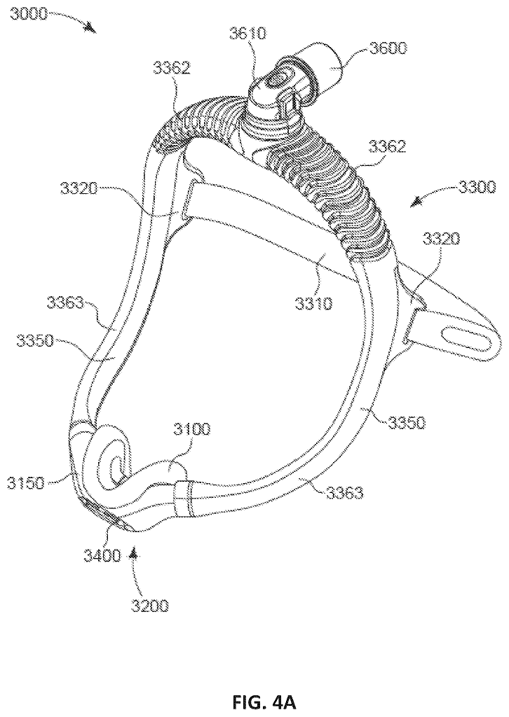

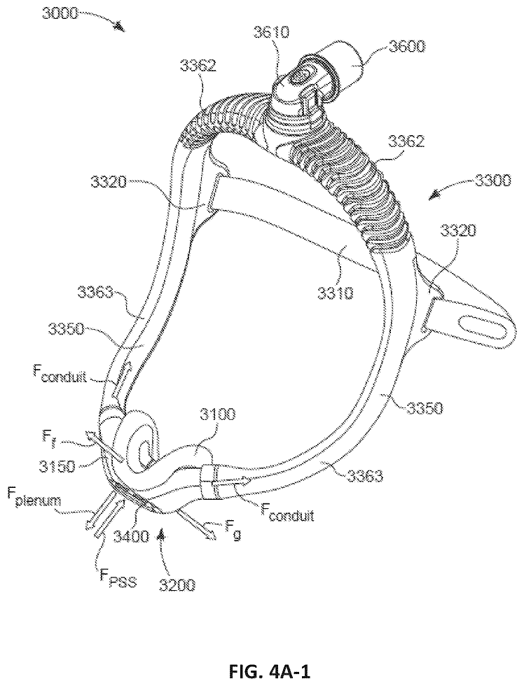

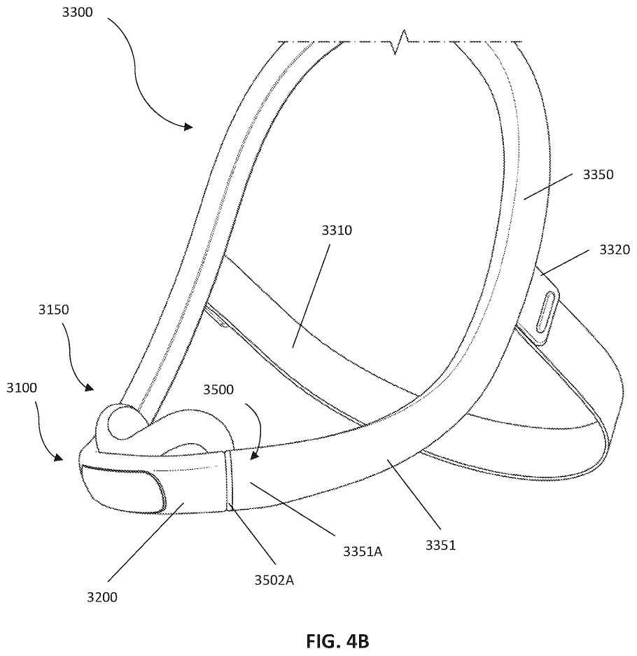

FIG. 4A shows a patient interface having conduit headgear, in accordance with one form of the present technology.

FIG. 4A-1 shows forces acting on the patient interface of FIG. 4, while in use.

FIG. 4B shows a patient interface having conduit headgear, in accordance with one form of the present technology.

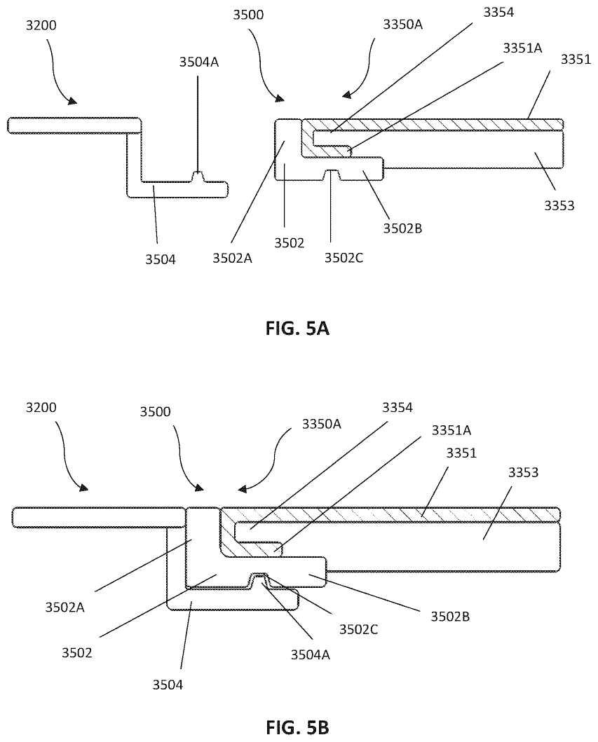

FIG. 5A shows a cross-sectional partial view of an inferior end of a conduit tube for conduit headgear and a plenum chamber prior to connection, in accordance with a first form of the present technology.

FIG. 5B shows a cross-sectional partial view of the inferior end of the conduit tube for conduit headgear and the plenum chamber of FIG. 5A, after connection.

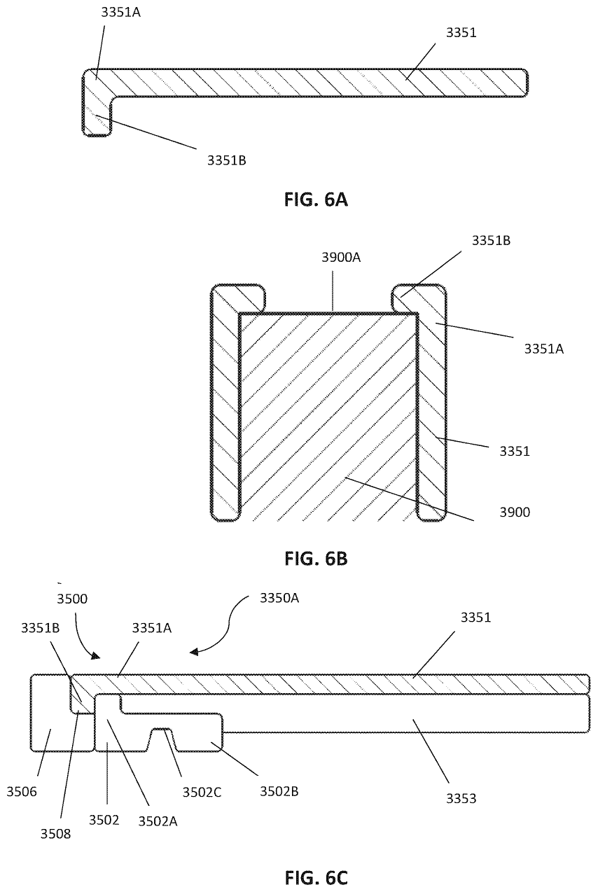

FIG. 6A shows a side view of an end of a textile sleeve for a conduit tube for conduit headgear, in accordance with a second form of the present technology.

FIG. 6B shows a schematic of the manufacture of the end of the textile sleeve of FIG. 6A.

FIG. 6C shows a cross-sectional partial view of an inferior end of a conduit tube for conduit headgear including the textile sleeve of FIG. 6A.

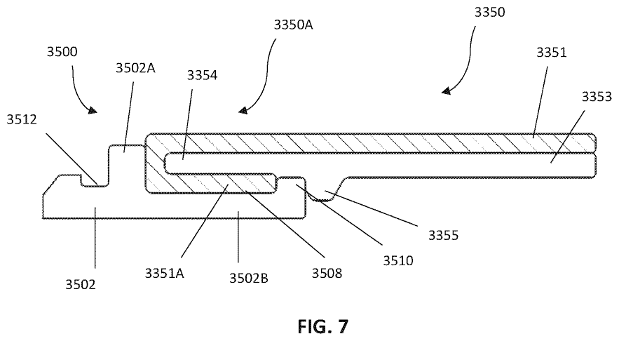

FIG. 7 shows a cross-sectional partial view of an inferior end of a conduit tube for conduit headgear, in accordance with a third form of the present technology.

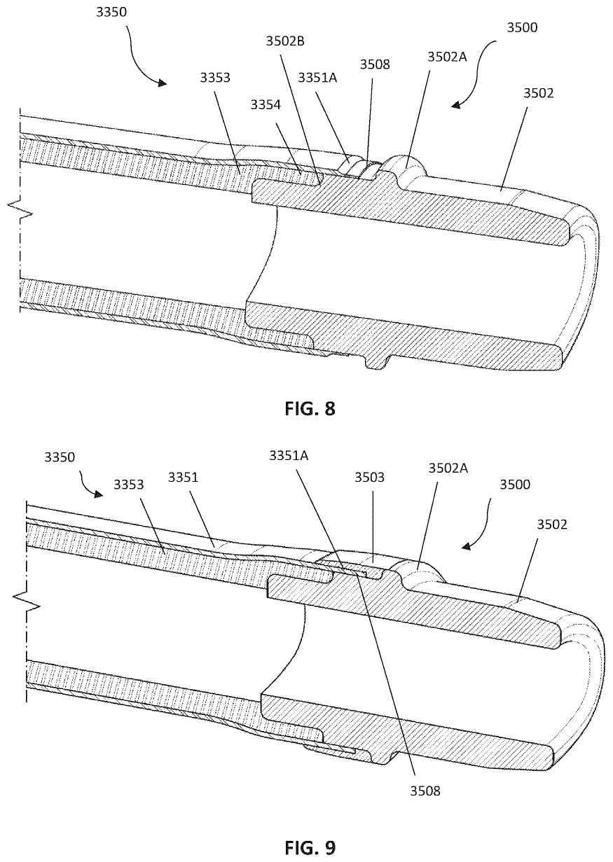

FIG. 8 shows a cross-sectional partial view of an inferior end of a conduit tube for conduit headgear, in accordance with a fourth form of the present technology.

FIG. 9 shows a cross-sectional partial view of the inferior end of the conduit tube for conduit headgear, in accordance with a fifth form of the present technology.

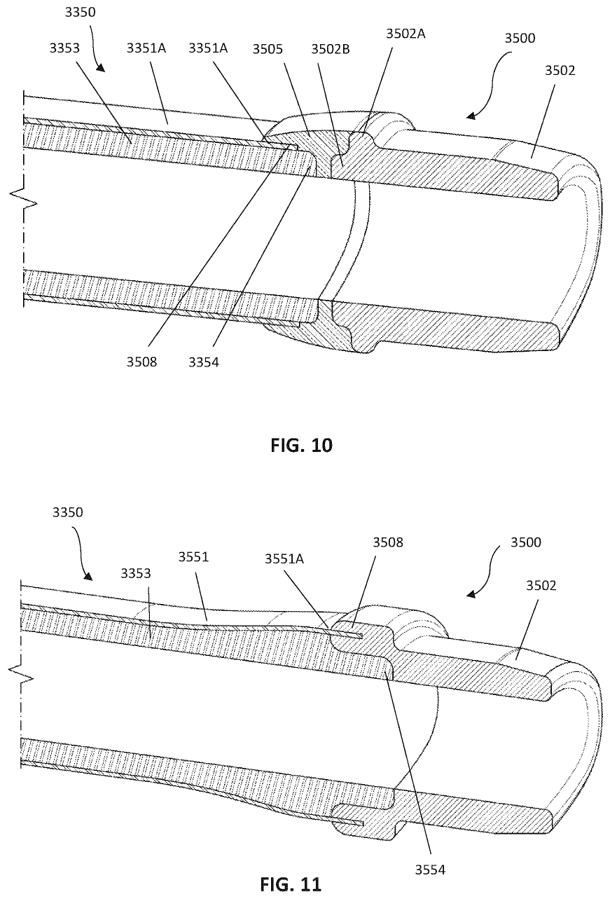

FIG. 10 shows a cross-sectional partial view of the inferior end of the conduit tube for conduit headgear, in accordance with a sixth form of the present technology.

FIG. 11 shows a cross-sectional partial view of the inferior end of the conduit tube for conduit headgear, in accordance with a sixth form of the present technology

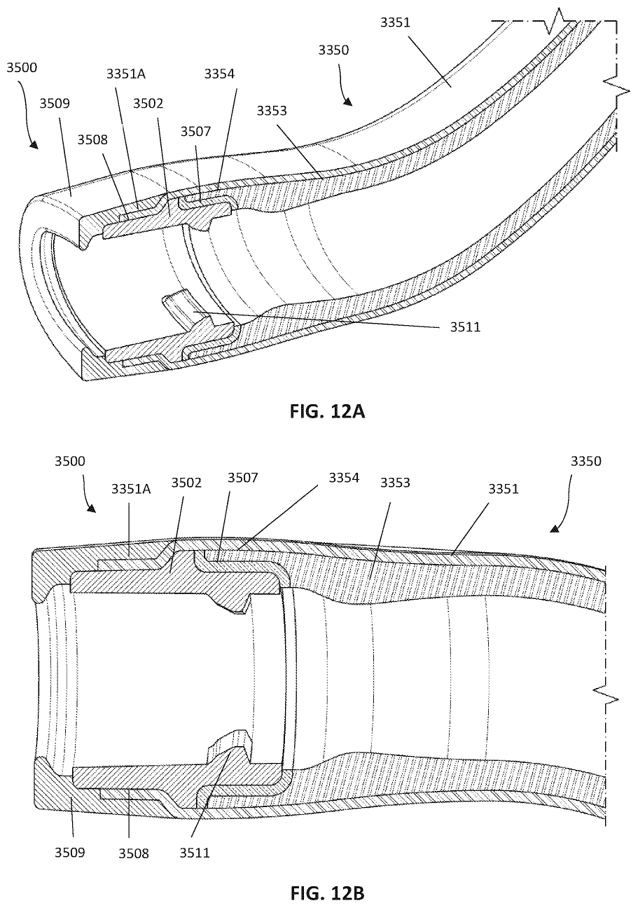

FIG. 12A shows a cross-sectional partial view of the inferior end of the conduit tube for conduit headgear, in accordance with a seventh form of the present technology.

FIG. 12B shows a further cross-sectional partial view of the inferior end of the conduit tube of FIG. 12A.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIGS. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 4A, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components (e.g., a cushion module 3150 together with the seal-forming structure 3100) in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH₂O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.1.7 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 4A, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of positioning and stabilising structures may be shown in FIGS. 3A and 4A. Examples of the forces applied by, for example, the positioning and stabilising structure of FIG. 4 may be shown in FIG. 4A-1.

In one form the positioning and stabilising structure 3300 provides a retention force at least sufficient to overcome the effect of the positive pressure in the plenum chamber 3200 to lift off the face (i.e., Fplenum).

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. suitable for a small sized head, but not a large sized head.

### 5.3.3.1 Conduit Headgear

### 5.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 4, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIGS. 4A, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap 3310 forms part of the positioning and stabilising structure 3300 and is positioned on the other side of the patient's head in use (e.g. across the other region) to assist in securing the patient interface 3000 on the patient's head. For example, the tube 3350 and the strap 3310 may each be under tension in use in order to assist in maintaining the seal-forming structure 3100 in a sealing position.

In one form, the tube 3350 may be at least partially extensible so that the tube 3350 and the strap 3310 may adjust substantially equal lengths when worn by a patient. This may allow for substantially symmetrical adjustments between the tube 3350 and the strap 3310 so that the seal-forming structure remains substantially in the middle.

In the form of the technology shown in FIG. 4, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

The tubes 3350 may be formed from a flexible material, such as an elastomer, e.g. silicone, thermoplastic elastomer (TPE) or thermoplastic polyurethane (TPU), and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 4A, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

Conduits forming part of the positioning and stabilising structure 3300, like headgear straps, may provide a force that contributes to the positioning and stabilising force FPSS. As illustrated in FIG. 4A-1, the positioning and stabilising force FPSS may be the resultant force from the various forces of the different elements of the positioning and stabilising structure 3300. For example, each conduit may provide a force Fconduit directed in the posterior and respective lateral direction in order to hold the seal-forming structure 3100 against the patient's face (into the upper lip and sealing under the nose) and oppose the effect of the positive pressure in the plenum chamber 3200 to lift off the face (i.e., Fplenum). The force Fconduit directed may also be directed at least partially in the superior direction in order to overcome the gravitational force Fg.

In some forms, the conduits may provide a force directed into the patient's head when the conduits are filled with pressurized air. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

The position of the patient's head may also change the gripping force of the conduits. For example, if the patient is sleeping on his side, the weight of the patient's head may compress one conduit, and the other conduit (e.g., the lateral portion not between the patient's head and a sleeping surface, like a pillow) may additionally expand in order to keep substantially the same flow rate of pressurized air.

Referring again to FIG. 4A-1, the gravitational force Fg may be opposed by a frictional force Ff, which may act in a direction directly opposite of the gravitational force Fg. As gravity pulls the seal-forming structure 3100 and the plenum chamber 3200 in the inferior direction, the frictional force Ff would act in the superior direction (e.g., against a patient's face). For example, the patient may experience the frictional force Ff against his lip superior (and/or other surfaces of the patient's face in contact with the seal-forming structure 3100) in order to oppose the motion in the inferior direction (which may help to stabilising the cushion in place). Although the frictional force Ff is shown specifically opposing the gravitational force Fg of the seal-forming structure 3100 and the plenum chamber 3200, components of an overall frictional force (not shown) would also oppose the gravitational force Fg associated with the positioning and stabilising structure 3300 and any other portions of the patient interface 3000. A force of friction can act along any place where the patient interface 3000 contacts the patient's skin (or hair). The frictional force Ff extends in the opposite direction of the gravitational force Fg and along the patient's skin (or hair).

In some forms, the sum of the various forces may equal zero so that the patient interface 3000 is at equilibrium (e.g., not moving along the patient's face while in use). Specifically, the gravitational force Fg and the blowout force Fplenum tend to move the seal-forming structure 3100 away from the desired sealing position. The positioning and stabilising force FPSS is applied in order to counteract the gravitational force Fg and the blowout force Fplenum (as well as any frictional forces Ff) and keep the seal-forming structure 3100 properly situated. Although the positioning and stabilising force FPSS may exceed the sum of the gravitational force Fg and the blowout force Fplenum (with any additional positioning and stabilising force FPSS being balanced by reaction force from the patient's head acting on the portions of patient interface 3000) and still maintain the seal-forming structure 3100 in an appropriate sealing position, patient comfort may be sacrificed. Maximum patient comfort may be achieved when the net force on the patient interface 3000 is zero and the positioning and stabilising force FPSS is exactly strong enough to achieve this. In some examples the positioning and stabilising structure 3300 may be adjustable such that when fitted the positioning and stabilising force FPSS is greater than required to exactly balance the gravitational force Fg and the blowout force Fplenum to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force FPSS necessary to achieve equilibrium

### 5.3.3.1.2 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 4A comprises tubes 3350, the superior portions of which comprise extendable tube sections each in the form of an extendable concertina structure 3362.

In some forms, the extendable concertina structure 3362 may be formed as a series of ridges and grooves on the surface of the tubes 3350. The concertina structure 3362 may be biased toward a retracted position, and may move to an expanded position when the patient dons the positioning and stabilising structure 3300. Because portions of the tubes 3350 may be substantially inextensible (e.g., non-extendable tube sections 3363), the concertina structures 3362 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3362. Alternatively, the tubes 3350 may be manufactured in multiple sizes (e.g., small, medium, large). The patient may select a length that most closely conforms to their head, and the concertina structures 3362 may make small adjustments in order to tailor the fit to the individual patient.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connects to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal forming structure 3100. The tubes 3350 may contact the patient's face below the patient's cheekbones, which may be more comfortable than contact on the patient's cheekbones and may avoid excessively obscuring the patient's peripheral vision.

### 5.3.3.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 4A, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.3.1.4 Headgear Tube Fluid Connections

The two tubes 3350 are fluidly connected at their inferior ends to the plenum chamber 3200 and at their superior ends to the connection port 3600. In certain forms of the technology, the connection between the tubes 3350 and the plenum chamber 3200 and/or connection port 3600 is achieved by a conduit connection structure 3500.

The conduit connection structure 3500 may be configured to enable the patient to easily connect the tubes 3350 to the plenum chamber 3200 and/or connection port 3600 in a reliable manner. The tubes 3350 and plenum chamber 3200 and/or connection port 3600 may be configured to provide tactile and/or audible feedback in the form of a ‘re-assuring click’ or a similar sound, so that the patient may easily know that each tube 3350 has been correctly connected. In one form, the tubes 3350 are formed from a silicone, TPE, or TPU, textile material or a blend thereof and the inferior end 3350A of each of the tubes 3350 is overmolded to the connector 3502 made, for example, from polypropylene, polycarbonate, nylon or the like.

The conduit connection structure 3500 on each tube 3350 may include a female mating feature 3502 configured to connect with a male mating feature 3504 provided to the plenum chamber 3200 and/or connection port 3600. Alternatively, the conduit connection structure 3500 on each tube 3350 may comprise a male mating feature configured to connect to a female mating feature on the plenum chamber 3200 and/or connection port 3600. In other examples the tubes 3350 may each comprise a male or female connector formed from a flexible material, such as silicone, TPE or TPU, for example the same material from which the tubes 3350 are formed.

In other examples the conduit connection structure 3500 may be in the form of a compression seal used to connect each tube 3350 to the plenum chamber 3200 and/or connection port 3600. For example, a resiliently flexible (e.g. silicone) tube 3350 may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350, the sealing flange may be urged against the join between the tubes 3350 and a circumferential surface around a port or connector of the plenum chamber 3200 to form or enhance a seal between the tube 3350 and plenum chamber 3200.

As previously discussed, the conduit tubes 3350 may be formed from a flexible material, such as an elastomer, e.g. silicone, TPE, TPU or other elastomer. For patient comfort, the conduit tubes 3350 may be covered with a textile. In some examples, this may be achieved with the use of a separate textile, provided as a tubular sleeve of a suitable material such as nylon, polyester or spandex of a blend thereof. This separate textile sleeve may be simply slid over the conduit tubes 3350 as required. In other examples, the textile may be provided in a sheet which may be wrapped around the conduit tubes 3350 and held in place with hook and loop material or the like.

In other examples, such as the patient interface shown in FIG. 4B, the inferior end of the textile sleeve 3351 may be integrated with the conduit tube 3350 using the conduit connection structure 3500 for a seamless finish at the inferior end of same, seen here, and the superior end of the textile sleeve 3351 may be integrated with a connector provided to the superior end (not visible) of the conduit tube 3350. This may provide a more aesthetically pleasing appearance for a conduit tube so configured in this way, compared to a conduit tube provided with a removable textile sleeve such as described above.

FIGS. 5A and 5B show a cross-sectional partial view of the inferior end 3350A of the conduit tube 3350 and the plenum chamber 3200, showing the conduit connection structure 3500 prior to connection (FIG. 5A) and after connection (FIG. 5B) of the conduit tube 3350 to the plenum chamber 3200.

The conduit connection structure 3500 is comprised of a suitable rigid plastics material, for example polycarbonate (PCB), polypropylene (PPE), nylon or a blend thereof, substantially in the form of a collar or ring circumscribing the perimeter of the end of the conduit tube 3350. In this example, the conduit connection structure 3500 is provided with an engagement portion in the form of a female mating feature or connector 3502 configured to connect with a complementary engagement portion in the form of a male mating feature or connector 3504 provided to the plenum chamber 3200. It will be appreciated that the connector 3504 may form part of an inlet port for the plenum chamber 3200. The respective female and male mating features 3502, 3504 may also circumscribe the perimeter of the conduit tube 3350 as shown here but may alternatively be provided as distinct prongs or similar protrusions, arranged equidistance around the collar.

As can be seen from the cross-sectional view, the conduit tube 3350 is formed from an inner tube 3353 or layer of a suitable elastomer material, such as silicone, TPE, TPU or another suitable elastomer, over which the textile sleeve 3351 is applied. The inferior end 3351A of the textile sleeve is folded over the mouth 3354 of the inner tube 3353 and contacts its interior surface proximate the mouth. The interior of the inner tube forms the path for air flow through said conduit tube 3350.

To keep the end 3351A of the textile sleeve 3351 out of the air path through the conduit tube 3350, the connector 3502 is configured such that it forms an L-shaped profile having a first arm 3502A, defined by the vertical portion of the connector, and a second arm 3502B, defined by a flange extending horizontally from the vertical portion. In this example, the inside surface of the L-shaped profile provides the contact surfaces with the mouth and interior surfaces of the inner tube. In use, this connector has an attachment region that receives and secures the inferior end 3351A of the textile sleeve between it (the connector) and the inner tube. The outside surface, i.e. the surface that faces the interior of the inner tube 3353, of the second arm 3502B is configured with a recess 3502C or groove or similar structure that receives a complementary ridge 3504A or protrusion or similar structure provided to the connector 3504 of the plenum chamber 3200. In this example, the outside surface of the first arm 3502A abuts and contacts the vertical portion of the connector 3504 of the plenum chamber 3200.

As best seen in FIG. 5B, the contact surface of the first arm 3502A bears against the mouth 3354 of the inner tube 3353 while the contact surface of the second arm 3502B, deliberately greater in length than the folded over end 3351A of the textile sleeve 3351, bears against the interior surface of the inner tube. As such, this traps the end 3351A of the textile sleeve 3351, preventing it from extending into the air path through the inner tube 3353. The mouth of the inner tube may be configured with a small recess or the like to better receive the folded over end 3351A of the textile sleeve.

As noted above, a portion of the outer surface of the connector 3502 forms part of the air path through the conduit tube 3350. Referring back to FIG. 4B, externally, when the conduit tube 3350 has been connected to the plenum chamber 3200 only the end of the first arm 3502A of the connector of the conduit connection structure 3500 is visible, and it can be seen that this provides a smooth-looking and flush transition at the juncture between the connector 3502 and the textile sleeve 3351.

During manufacture and/or assembly of the conduit tube 3350, a suitable adhesive or bonding agent may be applied to the attachment region defined by the inner surfaces of the first and second arms, 3502A and 3502B respectively, of the connector 3500 and/or mouth 3354 of the inner tube 3353 and/or end of the textile sleeve 3351. In the example shown, the adhesive is applied such that only the portion of the textile sleeve 3351 proximate and at the mouth 3354 of the inner tube 3353 is secured. This leaves the bulk of the remainder of the textile sleeve with some ability to move relative to the underlying inner tube. This may provide some useful give in the event the conduit tube 3350 is dragged against bedding or the like. In another example, the conduit connection structure 3500 and/or connector 3502 may be overmoulded to the mouth of the inner tube, trapping the end 3351A of the textile sleeve 3351.

Although only the inferior end 3350A of the conduit tube 3350 is shown here, it will be appreciated that the conduit connection structure provided to the superior end of the conduit tube may be configured in a similar manner to secure the textile sleeve 3351.

Another example of securing the textile sleeve 3351 to the inner tube 3353 using the conduit connection structure 3500 of the present invention is depicted in FIGS. 6A to 6C. This does so in such a way to place the end 3351A of the textile sleeve 3351 out of the air path of the conduit tube 3350. In this example, the textile sleeve 3351 is configured with a relatively rigid lip 3351B at its end 3351A, as shown in FIG. 6A. In this example, the material used to form the textile sleeve is substantially comprised of, or includes a blend of, thermofusible yarn. In some other examples, the textile sleeve may be comprised of two or more types of material; one type of material may be used for the body of the sleeve, and a thermofusible yarn used for the ends of the sleeve.

Configuring the textile sleeve 3351 with the lip 3351B may be achieved as shown in FIG. 6B, by placing the textile sleeve over a tubular-like jig 3900. The cross-sectional dimensions of the jig 3900 are such that the textile sleeve 3351 needs to be stretched over the same. By only partially inserting the jig 3900 into the textile sleeve 3351 (or by only partially pulling the textile sleeve 3351 onto the jig 3900), the end of same bears against the top surface 3900A of the jig 3900. The jig 3900 may then be heated to a sufficient temperature to fuse the end of the sleeve which, as it cools, forms the lip 3351B about the edges of the jig 3900.

FIG. 6C shows the conduit connection structure 3500 of the inferior end 3350A of the conduit tube in its assembled form. In this example, the connector 3502 of the conduit connection structure 3500 is a collar configured with a substantially L-shaped cross-sectional profile as discussed in respect of the embodiment of FIGS. 5A and 5B, with the inner side of the first arm 3502A being the contact surface bearing against and contacting the mouth 3354 of the inner tube 3353 while the inner side of the second arm 3502B is the contact surface bearing against the interior surface of the inner tube. The outer side of the second arm 3502B is configured with a recess 3502C to receive a complementary feature of the connector provided to the plenum chamber (not shown in this view). In this example, the connector 3502 has been overmoulded to the mouth 3354 of the inner tube 3353. An end cap 3506, which in this example, like the connector 3502, is provided as a L-shaped profile and is fixed to the connector 3502 using adhesive or another bonding agent to the first arm 3502A. This forms the attachment region 3508 in the form of a groove or recess running the circumference of the juncture between the end cap 3506 and conduit connection structure 3500. This groove 3508 receives the lip 3351B of the textile sleeve 3351 while being out of the path of air flow through the inner tube 3353. Adhesive may be used to secure the lip 3351B to the attachment region of the conduit connection structure 3500.

Note that in FIG. 6C, the conduit connection structure 3500 is effectively configured from two components: the main portion of the connector 3502 and the end cap 3506, the groove 3508 receiving the lip 3351B of the textile sleeve 3351 being formed at the juncture between the respective end cap 3506 and connector 3502. However, in some examples, the end cap 3506 may be integral to the connector 3502, i.e. may be formed as a single one-piece component. This may provide for a more rigid conduit connection structure 3500.

In some examples, the conduit connection structure 3500 of FIG. 6C may also be used with a textile sleeve 3351 which has not been manufactured in accordance with FIG. 6B. Instead, the end 3351A of the textile sleeve is simply folded into the groove 3508 and secured using adhesive or another bonding agent.

Turning to FIG. 7, this shows another example of the conduit connection structure 3500 of the inferior end 3350A of the conduit tube 3350. As with previous examples described, the conduit connection structure 3500 has a connector 3502 which is configured with a first arm 3502A, abutting the mouth 3354 of the inner tube 3353, and a second arm 3502B, which contacts the interior surface of the inner tube. At the end of the second arm 3502B is provided a ridge 3510 which, in use, abuts a complementary ridge 3355 provided to the interior surface of the inner tube 3353. The attachment region 3508 is at least partially defined by the ridge and the first arm 3502A. During assembly, the inferior end 3351A of the sleeve 3351 may be folded over the mouth 3354 of the inner tube 3353 and the connector 3502 inserted therein and bonded to the attachment region using an appropriate adhesive. The respective ridges 3510, 3355 act to ensure that the inferior end 3351A of the sleeve does not extend into the path of air flow through the conduit tube 3350.

In some examples, a series of ridges may be provided to the second arm 3502B and the interior surface of the inner tube 3353; these may provide a clamping effect, trapping the end 3351A of the textile sleeve 3351 between them while ensuring that the end 3351A does not enter the path of air flow through the inner tube 3353.

In contrast to previous examples described, the conduit connection structure 3500 has a connector 3502 of FIG. 7 which is configured as a male mating feature, to be received by a female mating feature provided to the plenum chamber (not shown in this view). A recess 3512 is provided to the connector 3502 to receive the female mating feature (not shown). It should be appreciated that the principles of the conduit connection structure as herein described may be applied regardless of whether the connector is configured with male or female mating features.

An alternative example of the conduit connection structure 3500 used for a conduit tube 3350 is shown in FIG. 8. As with previous embodiments described, the inner tube 3353, formed from an appropriate silicone, TPE, TPU or other such elastomer. To assemble the conduit tube 3350, the inner tube 3353 is mated to the second arm 3502B of the connector 3502, the respective components being appropriately bonded together. As can be seen the inner surfaces about the mouth 3354 of the inner tube 3353 are suitably recessed in order to receive the connector 3502. The connector 3502 is configured as an injection moulding of suitable biocompatible plastics material such as polycarbonate, polyamide/nylon, or polybutylene terephthalate.

The textile sleeve 3351 is then added to cover the inner tube 3353. In some instances, an adhesive coating is applied to the inner tube 3353 to help secure the textile sleeve along the majority of its length. The inferior end 3351A of the textile sleeve 3351 may be left to at least partially overlap the second arm 3502B of the connector 3502. The region of the connector upstream of the first arm 3502A forms part of the attachment region 3508 of the connector 3502. This attachment region is external to the air path flowing through the conduit tube 3350. For a tidy finish, the inferior end 3351A of the textile sleeve 3351 may be heat-treated or welded to the attachment region using ultrasonic technology; this may also minimise any errant fibres from the textile sleeve 3351.

The example of the conduit connection structure 3500 is shown in FIG. 9, is similar in construction to that previously described in respect of FIG. 8. However, the manner in which the inferior end 3351A of the textile sleeve 3351 is secured to the attachment region 3508 differs. As can be seen in FIG. 9, the inferior end 3351A of the textile sleeve 3351 may be fixed in place relative to the attachment region 3508 of the connector 3502 through the overmoulding or application of a cuff/collar 3503 of a thermoplastic elastomer. This may provide another way providing an aesthetically pleasing finish to the conduit tube 3350 while providing a potentially more robust means of securing the inferior end 3351A of the textile sleeve 3351. It should be appreciated that in some instances, even though the cuff/collar 3503 may still be applied afterwards, ultrasonic welding or bonding of the inferior end 3351 of the textile sleeve 3351 to the attachment region 3508 may still occur, if preferred. In some instances, this approach may be conducive to the use of a lower profile cuff/collar, which in turn may result in a smaller connector 3502.

Another form of the technology is shown in FIG. 10, and in some respects is similar to the example of FIG. 6C, in that the portion of the conduit connection structure 3500 provided to the conduit tube 3350 3500 is composed of two parts; the first part being the connector 3502, which is suitably configured to engage with the plenum chamber (not shown). As with the previous embodiments described, this connector 3502 may be formed as an injection moulding from a suitable thermoplastic material, such as polycarbonate, polyamide/nylon, or polybutylene terephthalate. The second part is a collar 3505, which serves to bond the connector 3502 to the inner tube 3353. The collar 3505 includes the attachment region 3508 which secures the inferior end 3351A of the textile sleeve 3351. The collar 3505 may be formed from an adhesive silicone. This arrangement may be useful in that the mouth 3354 of the inner tube 3353 may be a less complex moulding, which is advantageous for efficiency of manufacture.

In one example, the steps of manufacturing the conduit tube 3350 of FIG. 10 may be to form the inner tube 3353, add the textile sleeve 3351, and then apply the collar 3505. Being composed as adhesive silicone shot, the collar 3505 bonds to the mouth 3354 of the inner tube 3353 and also traps and secures the inferior end 3351A of the textile sleeve 3351 between it and the exterior surface of the inner tube 3353. The connector 3502 may then be added so it can also bond to the collar 3505. During manufacture, an excess of adhesive silicone may be applied to the conduit tube 3350 via appropriate moulding techniques (for example by adding an overflow to an internal core mould) to ensure a complete bond is achieved about the perimeter of the mouth 3354 of the inner tube 3353. This technique may also be used to ensure a substantially smooth finish or transition across the inner surfaces of the inner tube 3353 and the connector 3502 that define the path for air flowing therethrough. This may reduce potential imperfections in which undesirable fungal mould, bacteria or general dirt may accumulate.

FIG. 11 illustrates another form of the conduit connection structure 3500 which, like that of FIG. 10, may be conducive for efficient moulding of the inner tube 3353 since the mouth 3354 in particular has a simpler construction. During manufacture of this form of conduit tube 3350, the inner tube is moulded and then overlaid with the textile sleeve 3351. It will be seen that the exterior surfaces of the mouth 3354 are recessed relative to the rest of the inner tube. This leaves the inferior end 3351A of the textile sleeve 3351 with an overhang. The connector 3502, a one-piece structure, in contrast to that of FIG. 10, includes the attachment region 3508 at its superior, i.e. upstream end. This is applied to the mouth 3354 of the inner tube 3353 as an overmould, the attachment region 3508 receiving and securing the inferior end 3351A of the textile sleeve 3351. During the manufacturing process, the application of the connector 3502 may not immediately follow the formation of the inner tube 3353 and its covering with the textile sleeve 3351; in some examples, the inner tube may be allowed to at least partially cure or otherwise set.

While in some examples the connector 3502 of FIG. 11 may be formed from the previously mentioned materials of polycarbonate, polyamide/nylon, or polybutylene terephthalate, in other examples, it may be moulded from a silicone of a relatively high Shore durometer to provide an appropriate amount of rigidity. In a non-limiting example, the silicone of the connector 3502 may be selected from a hardness from between approximately 40 to 95 Shore A, or in another example, selected a hardness from between approximately 10 to 80 Shore D.

Yet another form of the conduit connection structure 3500 is shown in FIGS. 12A and 12B. As with previous examples described, the conduit tube 3350 is formed from an inner tube of silicone which is overlaid with a textile sleeve 3351. In this particular example however, an adhesive silicone overmould 3507 is applied to the first 3502A and/or second 3502B arms of the connector 3502 and/or the inner surfaces of the mouth 3354. Although this overmould 3507 is shown as visibly forming part of the air path through the conduit tube 3350 in FIGS. 12A and 12B, in some examples this region of the overmould 3507 may be crushed between the connector 3502 and the mouth 3354 of the inner tube, such that it is not visible once the conduit tube 3350 has been assembled.

In this form of the technology, the attachment region 3508 is formed by the exterior surface of the connector 3502. This receives an overmould 3509 composed of a suitable thermoplastic polyurethane or thermoplastic elastomer. As such, the connector 3502 is essentially a two-part structure; the first part, the connector 3502, is configured with a clip structure 3511 on an interior surface. This clip structure 3511 keys in or otherwise engages with a complementary recess or structure provided to an appropriate surface of the plenum chamber (not shown). The second part is the overmould 3509 which secures the inferior end 3351A of the textile sleeve 3351 to the attachment region 3508 (in some examples, the inferior end 3351A may also be ultrasonically welded or otherwise bonded to the attachment region 3508). The overmould 3509 may also be configured to form part of a lip seal with the plenum chamber (not shown).

An advantage of this configuration is that the exterior surfaces of the respective conduit tube 3350 and the conduit connection structure 3500 are substantially continuous and smooth. A further advantage is that the inner tube 3353 and connector 3502 may be bonded together prior to the textile sleeve 3351 being added to the conduit tube. This may be of assistance in the assembly of the conduit tube.

### 5.3.3.2 Headgear Straps

In the example shown in FIG. 4A, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 4, the positioning and stabilising structure 3300 comprises a pair of tabs 3320. In use a strap 3310 may be connected between the tabs 3320. The strap 3310 may be sufficiently flexible to pass around the back of the patient's head and lie comfortably against the patient's head, even when under tension in use.

In some forms, the strap 3310 may be at least partially bifurcated with a slit provided to the centre portion to divide the strap 3310 into a superior section and an interior section. This may allow the patient to have a larger strap coverage on the posterior region of their head, which may assist in better anchoring the headgear to the patient's head since there is no inferior strap.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

The vent 3400 may be located in the plenum chamber 3200 as shown in FIG. 4. Alternatively, the vent 3400 may be located in a decoupling structure, e.g., a swivel such as the elbow 3610.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support 3700.

### 5.3.8 Anti-Asphyxia Valve

In one form, the patient interface 3000 includes an anti-asphyxia valve.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 Rpt Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000.

### 5.6 Glossary

For the purposes of the present technology disclosure, in certain forms of the present technology, one or more of the following definitions may apply. In other forms of the present technology, alternative definitions may apply.

### 5.6.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H2O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m2=1 millibar ~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

5.6.1.1 Materials & their Properties

Hardness: Refers to durometer or indentation hardness, which is a material property measured by indentation of an indentor (e.g., as measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.6.1.2 Mechanics

### Axes:

a. Neutral axis: An axis in the cross-section of a beam or plate along which there are no longitudinal stresses or strains.
b. Longitudinal axis: An axis extending along the length of a shape. The axis generally passes through a center of the shape.
c. Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Viscous: The ability of a material to resist flow.

Visco-elasticity: The ability of a material to display both elastic and viscous behaviour in deformation.

Yield: The situation when a material can no longer return back to its original geometry after deformation.

### 5.6.1.3 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

Thin structures:
a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

Thick structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.6.2 Anatomy

### 5.6.2.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

Anatomy of the skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.6.3 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.7 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.8 REFERENCE SIGNS LIST

| | |
|---|---|
| patient | 1000 |
| bed partner | 1100 |
| patient interface | 3000 |
| seal - forming structure | 3100 |
| cushion module | 3150 |
| plenum chamber | 3200 |
| positioning and stabilizing structure | 3300 |
| strap | 3310 |
| tab | 3320 |
| conduit tube | 3350 |
| inferior end of conduit tube | 3350A |
| superior end of conduit tube | 3350B |
| Sleeve for conduit tube | 3351 |
| inferior end (of sleeve) | 3351A |
| lip (of sleeve) | 3351B |
| inner tube | 3353 |
| mouth (of inner tube) | 3354 |
| ridge (of inner tube) | 3355 |
| extendable (concentina) tube sections | 3362 |
| non - extendable tube sections | 3363 |
| vent | 3400 |
| conduit connection structure | 3500 |
| connector (of conduit tube) | 3502 |
| first arm (of connector) | 3502A |
| second arm (of connector) | 3502B |
| recess (of connector) | 3502C |
| flange (of connector) | 3502D |
| cuff/collar (of connector) | 3503 |
| connector (of plenum chamber) | 3504 |
| collar (of connector) | 3505 |
| ridge (of connector of plenum chamber) | 3504A |
| end cap (of connector of conduit tube) | 3506 |
| overmould | 3507 |
| attachment region (of connector) | 3508 |
| overmould (of connector) | 3509 |
| ridge (of second arm of connector) | 3510 |
| clip structure (of connector) | 3511 |
| recess (of connector) | 3512 |
| connection port | 3600 |
| elbow | 3610 |
| forehead support | 3700 |
| jig | 3900 |
| top surface (of jig) | 3900A |
| RPT device | 4000 |
| air circuit | 4170 |
| humidifier | 5000 |

1. A positioning and stabilising structure to provide a force to hold a seal-forming structure in a therapeutically effective position on a patient's head, the seal-forming structure constructed and arranged to form a seal with a region of the patient's face surrounding an entrance to the patient's airways for sealed delivery of a flow of air at a therapeutic pressure of at least 6 cmH₂O above ambient air pressure throughout the patient's respirator cycle in use, the positioning and stabilising structure comprising:
at least one gas delivery tube to receive the flow of air from a connection port on top of the patient's head and an inferior end configured to deliver the flow of air to the entrance of the patient's airways via the seal-forming structure, the at least one gas delivery tube being constructed and arranged to contact, in use, at least a region of the patient's head superior to an otobasion superior of the patient's head, the at least one gas delivery tube comprising:
an inner tube comprising a tube wall defining a hollow interior for a flow path for air to flow from the connection port to the seal-forming structure, and wherein the inner tube is configured with an inferior end and a superior end;
an outer textile sleeve having an inferior end and a superior end; and
a conduit connection structure provided to at least one of the inferior and superior ends of the inner tube, wherein the conduit connection structure is configured to connect the inner tube to one of the connection port or seal-forming structure, wherein the conduit connection structure is configured with a first surface that forms part of the flow path through the hollow interior of the inner tube, and wherein the conduit connection structure is configured with a second surface away from the flow path through the hollow interior of the inner tube and forming at least a part of an attachment region;
wherein the attachment region is configured to receive one of the inferior and superior ends of the outer textile sleeve.

2. The positioning and stabilising structure as claimed in claim 1, wherein the conduit connection structure includes an engagement portion configured to engage with a complementary engagement portion provided to one of the connection port or seal-forming structure.

3. The positioning and stabilising structure as claimed in claim 1, wherein the conduit connection structure includes a first collar provided to one of the inferior end and superior end of the inner tube.

4. The positioning and stabilising structure as claimed in claim 3, wherein the first collar includes an inner side having a first contact surface and a second contact surface, and an outer side.

5. The positioning and stabilising structure as claimed in claim 4, wherein the first contact surface of the first collar is substantially perpendicular to the second contact surface of the first collar.

6. The positioning and stabilising structure as claimed in claim 4, wherein the first contact surface contacts a mouth of the inner tube and the second contact surface contacts an interior surface of the hollow interior of the inner tube.

7. The positioning and stabilising structure as claimed in claim 4, wherein one of the first contact surface and second contact surface contacts a second collar provided to the mouth of the inner tube.

8. The positioning and stabilising structure as claimed in claim 4, wherein the attachment region is formed by at least a portion of the second contact surface.

9. The positioning and stabilising structure as claimed in claim 7, wherein the attachment region is formed by at least a portion of the second collar.

10. The positioning and stabilising structure as claimed claim 4, wherein the attachment region is less than the length of the second contact surface.

11. The positioning and stabilising structure as claimed in claim 4, wherein the end of the second contact surface includes a ridge or protrusion.

12. The positioning and stabilising structure as claimed in claim 4, wherein the interior surface of the hollow interior of the inner tube includes a ridge or protrusion to abut that of the end of the second contact surface.

13. The positioning and stabilising structure as claimed in claim 1, wherein the inferior end of the textile sleeve is adhesively secured or otherwise bonded to the attachment region.

14. The positioning and stabilising structure as claimed in claim 3, wherein the inferior end of the textile sleeve is secured or otherwise bonded to the attachment region by overmoulding the first collar to one of the inferior end or superior end of the inner tube.

15. The positioning and stabilising structure as claimed in claim 3, wherein the inferior end of the textile sleeve is secured or otherwise bonded to the attachment region by overmoulding a second collar to one of the inferior end or superior end of the inner tube.

16. The positioning and stabilising structure as claimed in claim 4, wherein at least a portion of the outer side of the first collar is the first surface that forms at least part of the flow path through the hollow interior of the inner tube.

17. The positioning and stabilising structure as claimed in claim 1, wherein at least a portion of the first surface includes the engagement portion configured to engage with the complementary engagement portion provided to one of the connection port or seal-forming structure.

18. The positioning and stabilising structure as claimed claim 4, wherein the first collar includes an end cap.

19. The positioning and stabilising structure as claimed in claim 18, wherein the end cap is connected to the first collar on a surface opposing the first contact surface of the collar.

20. The positioning and stabilising structure as claimed in claim 18, wherein the attachment region is the juncture between the end cap and the first collar.

21. The positioning and stabilising structure as claimed claim 18, wherein the end cap is integrally formed with the first collar.

22. The positioning and stabilising structure as claimed in claim 15, wherein the juncture between the end cap and the first collar is a groove or recess.

23. The positioning and stabilising structure as claimed in claim 15, wherein the inferior end of the textile sleeve is secured or otherwise bonded to the attachment region by overmoulding the end cap to the first collar.

24. The positioning and stabilising structure as claimed in claim 1, wherein the conduit connection structure is provided to both the inferior end and superior end of the inner tube.

25. The positioning and stabilising structure as claimed in claim 1, wherein the conduit connection structure is comprised of one or more plastics material of polycarbonate (PCB), polypropylene (PPE), nylon or a blend thereof.

26. The positioning and stabilising structure as claimed in claim 1, wherein the inner tube is comprised of an elastomer.

27. The positioning and stabilising structure as claimed in claim 26, wherein the elastomer is comprised of silicone, thermoplastic elastomer (TPE), or thermoplastic polyurethane (TPU), or another elastomer material or a blend of same.

28. The positioning and stabilising structure as claimed in claim 1, wherein the outer textile sleeve is comprised of a thermofusible material.

29. The positioning and stabilising structure as claimed in claim 28, wherein the thermofusible material is a yarn.

30. The positioning and stabilising structure as claimed in claim 1, wherein one of the inferior end and superior end of the textile sleeve has been heat treated to form a lip.

31. The positioning and stabilising structure as claimed in claim 30, wherein the lip is received by the attachment region of the conduit connection structure.

32. The positioning and stabilising structure as claimed in claim 1, wherein both of the inferior end and superior end of the textile sleeve has been heat treated to form a lip.

33. A gas delivery tube for a positioning and stabilising structure, wherein the positioning and stabilising structure is configured with a connection port on top of a patient's head and a seal forming structure constructed and arranged to form a seal with a region of the patient's face surrounding an entrance to the patient's airways for sealed delivery of a flow of air at a therapeutic pressure of at least 6 cmH₂O above ambient air pressure throughout the patient's respirator cycle in use, wherein the gas delivery tube is configured to receive the flow of air from the connection port to the seal-forming structure, the gas delivery tube comprising:
an inner tube comprising a tube wall defining a hollow interior for a flow path for air to flow from the connection port to the seal-forming structure, and wherein the inner tube is configured with an inferior end and a superior end;
an outer textile sleeve having an inferior end and a superior end; and
a conduit connection structure provided to at least one of the inferior and superior ends of the inner tube, wherein the conduit connection structure is configured to connect the inner tube to one of the connection port or seal-forming structure, wherein the conduit connection structure is configured with a first surface that forms part of the flow path through the hollow interior of the inner tube, and wherein the conduit connection structure is configured with a second surface away from the flow path through the hollow interior of the inner tube and forming at least a portion of an attachment region;
wherein the attachment region is configured to receive one of the inferior and superior ends of the outer textile sleeve.
